(19) United States
(12) Patent Application Publication
(10) Pub. No.: US 2005/0245944 A1
(43) Pub. Date: Nov. 3, 2005

(54) Apparatus for facilitating delivery of at least one device to a target site in a body

(76) Inventor: Ali R. Rezai, Bratenhal, OH (US)

Correspondence Address: Tarolli Sundheim;Covell & Tummino, Suite 1111, 526 Superior Avenue, Cleveland, OH, 44114-1400, US

(21) Appl. No.: 10/502,631

(22) PCT Filed: Jan. 31, 2003
(86) PCT No.: PCT/US03/02875

§ 371 Date: May 26, 2005

Related U.S. Application Data
(60) Provisional application No. 60/353,695, filed on Feb. 1, 2002.

(51) Int. Cl. A61B 19/00

(52) U.S. Cl. 606/129

(57) ABSTRACT

An apparatus (10) for facilitating delivery of at least one device (100) to a target site (14) in a body (16) includes a tubular member (12) having a tubular side wall (40) with an inner surface (44) and an opposite outer surface (46). The inner surface defines an inner lumen (50) of the member (12) extending from a proximal end (30) of the member to a distal end (32) of the member. The member (12) is insertable into the body (16) to position the distal end (32) at a predetermined position relative to the target site. The apparatus (10) also includes at least one port (70) positioned between the inner surface (44) and the outer surface (46). The at least one port (70) extends from the proximal end (30) toward the distal end (32) and exits the side wall (40) at a predetermined position relative to the distal end.

### RELATED APPLICATIONS

This application claims the benefit of U.S. Provisional Patent Application No. 60/353,695, filed on Feb. 1, 2002.

### FIELD OF THE INVENTION

The present invention relates to an apparatus for facilitating introduction of at least one medical device to a target site in a body.

### BACKGROUND OF THE INVENTION

Cannulas are employed to facilitate access through openings in the skin for the insertion of medical devices, such as biopsy tools or electrical stimulation electrodes, into soft tissue and organs. The cannula is typically inserted through a puncture or opening in the skin and the medical device is inserted through a central opening or lumen of the cannula to the desired position in the body. Depending on the type of procedure, the cannula may then be removed, leaving the medical device implanted in the body, or the cannula and the medical device may both be removed.

Current delivery systems employ a cannula having a single lumen. These single lumen cannulas permit the introduction of a single medical device into the body. Use of such single lumen cannulas in stereotactic based medical procedures limits the ability of the surgeon performing the procedure to detect and treat possible complications that may arise during the procedure. For example, bleeding in the brain may occur while employing the use of a cannula during a neurosurgical procedure. Typically, such complications are detected based upon a neurological deficit or upon a post-procedural CT scan or MRI scan. It is, however, desirable to treat these complications hyperacutely, in order to help prevent a permanent neurological deficit.

An object of the present invention is to provide a cannula that promotes safety during a variety of surgical procedures. To meet this objective, the spirit of the present invention is to provide a cannula that allows for the delivery of a medical device to a target area of a patient, provides for diagnostic and physiologic monitoring at the target area, and allows for the delivery of agents to the target area.

### SUMMARY OF THE INVENTION

The present invention relates to an apparatus for facilitating introduction of at least one medical device to a target site in a body. The apparatus includes a tubular side wall forming an inner lumen through which a first medical device is delivered to the target site. The apparatus also includes a plurality of ports, located in the side wall, that provide access to the target site. The ports provide access to the target site for additional medical devices that aid in the detection and treatment of complications that may arise during the introduction of the first medical device.

In one embodiment, the present invention relates to an apparatus for delivering a stimulation electrode to a target site in a human brain during a stereotactic neurological surgical procedure. In this embodiment, the electrode is delivered through the inner lumen and the ports allow for the use of medical devices that aid in the detection and hyperacute treatment of complications that may arise during the surgical procedure in order to help prevent a permanent neurological deficit.

The present invention also relates to an apparatus for facilitating delivery of a device to a target site in a body includes a tubular member having a tubular side wall with an inner surface and an opposite outer surface. The inner surface defines an inner lumen of the member extending from a proximal end of the member to a distal end of the member. The member is insertable into the body to position the distal end at a predetermined position relative to the target site. The apparatus also includes at least one port positioned between the inner surface and the outer surface. The at least one port extends from the proximal end toward the distal end and exits the side wall at a predetermined position relative to the distal end.

The present invention also relates to an apparatus including an elongate member having a side wall and at least one inner lumen extending from a proximal end of the member to a distal end of the member. The member is insertable into a body to position the distal end at a predetermined position relative to a target site in the body. The apparatus also includes at least one port in the form of a longitudinal bore in the side wall. The at least one port extends from the proximal end toward the distal end and exits the side wall at a predetermined position relative to the distal end.

The present invention also relates to an apparatus including an elongate tubular member having a longitudinal axis. The member has an axially extending cylindrical side wall with an inner surface defining a cylindrical inner lumen extending from a proximal end of the member to a distal end of the member. The member is insertable into a body to position the distal end at a predetermined position relative to a target site in the body. The apparatus also includes at least one port extending through the side wall in a direction parallel to the axis from the proximal end toward the distal end. The at least one port exits the side wall at a predetermined position relative to the distal end.

The apparatus further relates to an apparatus including an elongate tubular member having a longitudinal axis. The member includes a cylindrical inner sleeve positioned within and extending coaxially with a cylindrical outer sleeve. The inner sleeve has an inner surface defining a cylindrical inner lumen extending from a proximal end of the member to a distal end of the member. The member is insertable into the body to position the distal end at a predetermined position relative to the target site. The apparatus also includes at least one port defined between an inner surface of the outer sleeve and an outer surface of the inner sleeve. The at least one port extends from the proximal end to a predetermined position relative to the distal end.

The present invention still further relates to an apparatus including an elongate cylindrical side wall defining a central lumen extending along an axis from a proximal end of the side wall to a distal end of the side wall. The apparatus also includes at least one port extending within the side wall in a direction parallel to the axis from the proximal end toward the distal end. The at least one port exits the side wall at a predetermined position relative to the distal end.

### BRIEF DESCRIPTION OF THE DRAWINGS

The foregoing and other features of the invention will become apparent to one skilled in the art to which the invention relates upon consideration of the following description of the invention with reference to the accompanying drawings, in which:

FIG. 1 is a schematic illustration of an apparatus for facilitating delivery of a device to a target site in a body, according to a first embodiment of the present invention;

FIG. 2 is a magnified side view of the apparatus of FIG. 1;

FIG. 3 is a top view of the apparatus of FIG. 2;

FIG. 4 is a bottom view of the apparatus of FIG. 2;

FIG. 5 is a fractured perspective illustration of an exemplary use of the apparatus of FIGS. 1-4;

FIG. 6 is a magnified side view of an apparatus for facilitating delivery of a device to a target site in a body, according to a second embodiment of the present invention;

FIG. 7 is a magnified sectional view of an apparatus for facilitating delivery of a device to a target site in a body, according to a third embodiment of the present invention;

FIG. 8A is a sectional view taken generally along line 8A-8A in FIG. 7;

FIG. 8B is a sectional view taken generally along line 8B-8B in FIG. 7; and

FIG. 9 is a magnified side view of an apparatus for facilitating delivery of a device to a target site in a body, according to a fourth embodiment of the present invention.

### DETAILED DESCRIPTION OF THE INVENTION

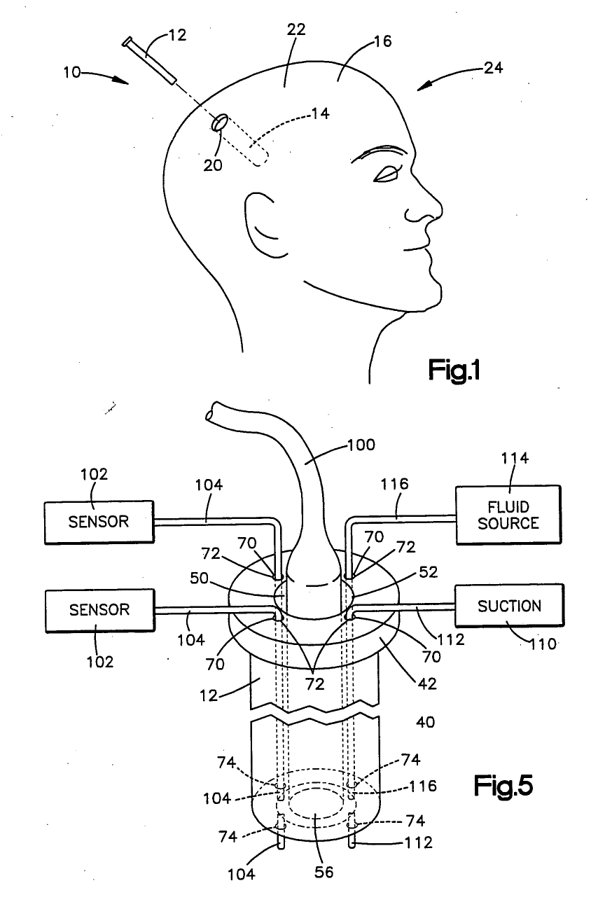

The present invention relates to an apparatus for facilitating delivery of a device to a target site in a body. More specifically, referring to FIG. 1, the apparatus 10 of the present invention is a ported cannula 12 for facilitating the introduction of at least one medical device to a target site 14 in a body 16. As shown in FIG. 1, according to a first embodiment of the present invention, the cannula 12 is employed to facilitate introduction of medical devices to the brain (not shown) through a burr hole 20 in the head 22 of a patient 24.

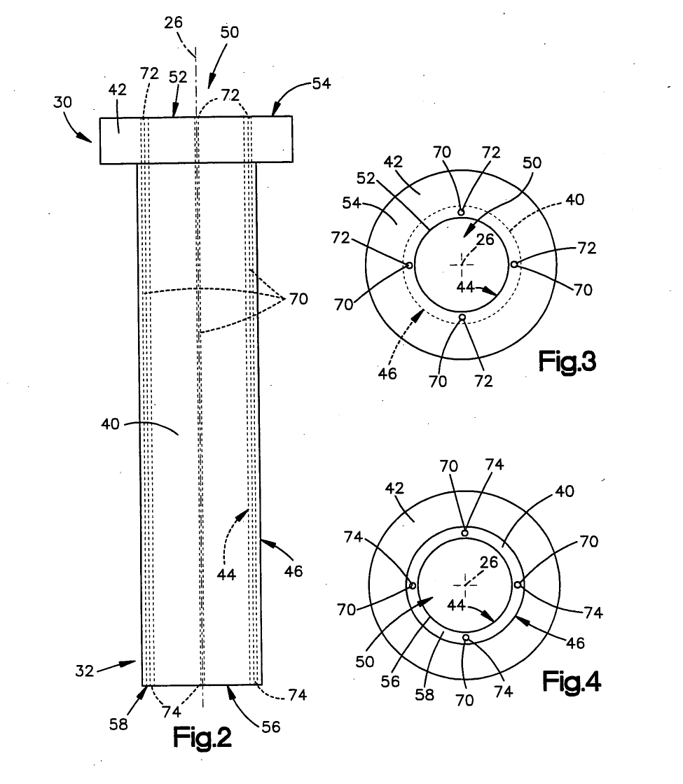

Referring to FIGS. 2-4, the cannula 12 has a generally elongate tubular construction with a proximal end 30 and a distal end 32. The cannula 12 has a longitudinal axis 26 and a cylindrical side wall 40 centered on the axis. The cannula 12 may also include an annular collar 42 at the proximal end 30 of the cannula. The collar 42 may be adapted to cooperate with stereotactic positioning equipment (not shown) for positioning the cannula 12 relative to the target site 14 (see FIG. 1).

The side wall 40 has an inner surface 44 and an opposite outer surface 46. The inner surface 44 defines an inner lumen 50 of the cannula 12. The lumen 50 is centered on the axis 26 and extends through the cannula 12 from the proximal end 30 to the distal end 32. The lumen 50 includes an entrance orifice 52 formed on an end surface 54 at the proximal end 30 of the cannula 12. The lumen 50 also includes an exit orifice 56 formed on an end surface 58 at the distal end 32 of the cannula 12.

The cannula 12 also includes a plurality of ports 70 that are spaced about the axis 26 along the circumference of the side wall 40. In the embodiment illustrated in FIGS. 2-4, the cannula 12 includes four ports 70 spaced radially, approximately ninety degrees apart, about the axis 26. It will be appreciated, however, that the ports 70 may be spaced differently and that the cannula 12 could include a greater or lesser number of ports 70. The ports 70 extend longitudinally through the side wall 40 in a direction parallel to the axis 26. Each of the ports 70 includes an entrance orifice 72 formed on the end surface 54 at the proximal end 30 and an exit orifice 74 formed on the end surface 58 at the distal end 32.

The cannula 12 may be constructed in a variety of manners. For example, the cannula 12 may have a stainless steel construction in which the cannula is machined or otherwise formed from stock material. Alternatively, the cannula 12 may have a polymer construction in which the cannula is molded or otherwise formed from raw materials. As a further alternative, the cannula may have any construction suited to provide the performance required for its intended use.

In the embodiment illustrated in FIGS. 2-4, the side wall 40 has a solid construction, with the lumen 50 and the ports 70 being bored or otherwise formed therethrough. For example, the cannula 12 may be formed from tube stock, with the lumen 50 comprising the central opening in the tube stock and the ports 70 being drilled or otherwise bored through the side wall 40. Other constructions of the cannula 12 are described below.

The cannula 12 may also have a size that varies in accordance with its intended use. For example, in the illustrated embodiment wherein the cannula 12 is used to deliver an electrode to a patient's brain, the side wall 40 of the cannula 12 may have an outside diameter as small as 5-10 millimeters, or smaller. The length of the side wall 40 may be about 10-18 centimeters. The lumen 50 and/or ports 70 may be as small as one millimeter in diameter or smaller. The cannula 12 could, of course, be smaller or larger in diameter and/or length, depending on the type and location of the surgical procedure being performed.

In this description of the present invention, the operation of the cannula 12 will be described in an exemplary use in which the cannula is used during a neurosurgical procedure to introduce a stimulating electrode into the brain of a patient. It will be appreciated, however, that the cannula 12 of the present invention may be used in a variety of neurosurgical procedures to introduce instruments or medical devices, such as biopsy tools, microelectrodes, or lesioning electrodes, to the brain. It will further be appreciated that the cannula 12 of the present invention may be used to introduce instruments or medical devices other parts of the patient's body during the course of a variety of surgical procedures.

According to the present invention, the ports 70 of the cannula 12 provide access to the target site 14 in addition to the access provided by the lumen 50. This allows for the use of devices that may aid in the delivery of the stimulating electrode into the brain during the neurosurgical procedure. Such devices, for example, may provide a means by which to detect and provide immediate treatment of complications that may arise during the procedure. These devices may also aid in ascertaining whether proper delivery and/or placement of the electrode has been achieved.

In an exemplary configuration shown in FIG. 5, a stimulating electrode 100 is insertable into the lumen 50 through the entrance orifice 52. Sensors 102 have leads 104 inserted into respective ones of the ports 70 through their respective entrance orifices 72. A suction source 110 has a suction conduit 112, such as a stylet, inserted into yet another of the ports 70 through its entrance orifice 72. A fluid source 114 has a delivery conduit 116, such as a stylet, inserted into the remaining port 70 through its entrance orifice 72. As shown in the fractured view of FIG. 5, the electrode 100 may exit the cannula 12 through the exit orifice 56 of the lumen 50. The leads 104 and the conduits 112 and 116 may also exit the cannula 12 through the exit orifices 74 in their respective ports 70.

The sensors 102, positioned as illustrated in FIG. 5, are adapted to help detect the occurrence of complications that may arise during the neurosurgical procedure. The sensors 102 may also aid in guiding the cannula 12 and/or electrode 100 to the target area. the sensors 102 may further help ensure proper placement of the electrode 100 in the target area. The sensors 102 may be of a variety of available types. For example, the sensors 102 may comprise a fiberoptic transducer operable to provide a visual image of the target area. As another example, the sensors 102 may comprise an infrared transducer operable to provide an infrared image of the target area. As a further example, the sensors 102 may comprise pressure transducers, pH probes, ultrasound probes, or any other device suited to sense a desired condition at the target area via one or more of the ports 70.

The suction source 110 provides a means by which to draw or suction out unwanted fluids, such as blood or irrigation fluids, from the target area. The fluid source 114 may be used to inject fluids, such as saline solution, for irrigating the target area. The fluid source 114 may also be used to inject medications or agents, such as thrombotic agents, in the event that a complication, such as bleeding, is encountered. Once the electrode 100 is delivered to the target site, the cannula 12, and the associated leads 104 and conduits 112 and 116, may be removed, thus leaving the electrode implanted at the target area.

Those skilled in the art will appreciate that the cannula 12 of the present invention provides a means for detection and treatment of complications that may arise during a stereotactic procedure, such as the neurosurgical procedure described above. Because the cannula 12 of the present invention provides sensing capabilities at the target site, complications may be detected immediately during the procedure and treated promptly, which may help eliminate or reduce any lasting effects.

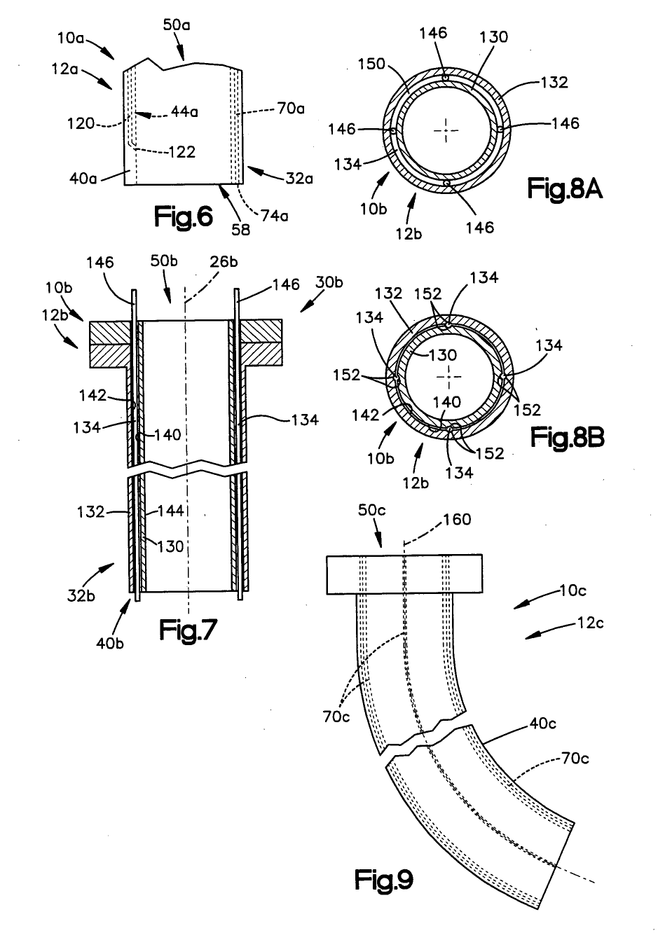

A second embodiment of the present invention is illustrated in FIG. 6. The second embodiment of the invention is similar to the first embodiment of the invention illustrated in FIGS. 1-5. Accordingly, numerals similar to those of FIGS. 1-5 will be utilized in FIG. 6 to identify similar components, the suffix letter “a” being associated with the numerals of FIG. 6 to avoid confusion.

Referring to FIG. 6, the apparatus 10a of the second embodiment comprises a cannula 12a. FIG. 6 illustrates the distal end 32a of the cannula 12a. The cannula 12a includes one port 70a that, like in the first embodiment, exits the side wall 40a at an exit orifice 74a on a surface 58a at the distal end 32a. The cannula 12a also includes a port 120 that exits the side wall 40a through an exit orifice 122 on the inner surface 44a of the side wall. In this configuration, a device, such as a lead or conduit (not shown), may access the lumen 50a and thus provide sensing, fluid delivery, or suction at a particular location along the length of the cannula 12a. Although the exit orifice 122 is illustrated near the distal end 32a of the cannula 12a in FIG. 6, it will be appreciated that the exit orifice of the second embodiment may be located at any desired location along the length of the cannula.

A third embodiment of the present invention is illustrated in FIG. 7. The third embodiment of the invention is similar to the first embodiment of the invention illustrated in FIGS. 1-5. Accordingly, numerals similar to those of FIGS. 1-5 will be utilized in FIG. 7 to identify similar components, the suffix letter “b” being associated with the numerals of FIG. 7 to avoid confusion.

Referring to FIG. 7, the apparatus 10b of the second embodiment comprises a cannula 12b. According to the third embodiment, the side wall 40b of the cannula 12b includes an inner sleeve 130 and an outer sleeve 132. The inner sleeve 130 is positioned within and extends coaxially with the outer sleeve 132 along the axis 26b. The cannula 12b includes ports 134 that are defined by an annular space between an outer surface 140 of the inner sleeve 130 and an inner surface 142 of the outer sleeve 132. The lumen 50b is defined by an inner surface 144 of the inner sleeve.

Referring to FIG. 8A, in one configuration in accordance with the third embodiment of the present invention, the ports 134 comprise an annular clearance 150 formed between the inner sleeve 130 and the outer sleeve 132. In this configuration, leads or conduits, indicated at 146, are positioned within the clearance 150 and extend from the proximal end 30b to the distal end 32b (see FIG. 7). Those skilled in the art will appreciate that the configuration of FIG. 8A is advantageous in that it is flexible to accept a greater number of leads or conduits 146 and that the leads or conduits may be adjusted to a desired annular position within the clearance 150.

Referring to FIG. 8B, in another configuration in accordance with the third embodiment of the present invention, the ports 134 are formed by opposing semi-cylindrical grooves 152 in the outer surface 140 of the inner sleeve 130 and the inner surface 142 of the outer sleeve 132. This allows for a closer fit or tolerance between the remaining portions of the inner and outer sleeves 130 and 132. In this configuration, leads or conduits (not shown for clarity), are positioned within the ports 134 formed by the opposing grooves 152 and extend from the proximal end 30b to the distal end 32b (see FIG. 7). Those skilled in the art will appreciate that the configuration of FIG. 8B is advantageous in that a close or tight fit may be provided between the inner and outer sleeves and that the leads or conduits may be better held in position within the ports 134.

A fourth embodiment of the present invention is illustrated in FIG. 9. The fourth embodiment of the invention is similar to the embodiments of the invention illustrated in FIGS. 1-8B. Accordingly, numerals similar to those of FIGS. 1-8B will be utilized in FIG. 9 to identify similar components, the suffix letter “c” being associated with the numerals of FIG. 9 to avoid confusion.

Referring to FIG. 9, the apparatus 10c of the third embodiment comprises a cannula 12c. The cannula 12c of the fourth embodiment may have a construction similar to any of the first three embodiments illustrated in FIGS. 1-8B. The cannula 12c, however, has a curved configuration extending along a curved axis 160. The side wall 40c, and thus the lumen 50c and the ports 70c, follow or extend parallel to this curved axis 160. The cannula 12b of the fourth embodiment may be advantageous in a situation in which a curved path must be followed in order to reach a target site in a body.

The present invention disclosed herein provides a cannula that allows for the delivery of a medical device to a target area of a patient, provides for diagnostic and physiologic monitoring at the target area, and allows for the delivery of agents to the target area. The cannula of the present invention may thus promote safety during a variety of surgical procedures.

From the above description of the invention, those skilled in the art will perceive improvements, changes and modifications in the invention. For example, although the tubular configuration of the cannula has been described herein as being generally cylindrical, it will be appreciated that the cannula may have alternative tubular configurations, such as elliptical, rectangular, or polygonal. Also, although the illustrated embodiments depict a cannula having a single lumen, it will be appreciated that any of the embodiments disclosed herein may include multiple lumens. In this instance, the ports may be spaced radially about the multiple lumens or at any other desired position relative to the lumens. Such improvements, changes and modifications within the skill of the art are intended to be covered by the appended claims.

1. An apparatus for facilitating delivery of a device to a target site in a body, said apparatus comprising:
a tubular member having a tubular side wall with an inner surface and an opposite outer surface, said inner surface defining an inner lumen of said member extending from a proximal end of said member to a distal end of said member, said member being insertable into the body to position said distal end at a predetermined position relative to the target site; and
at least one port positioned between said inner surface and said outer surface and extending from said proximal end toward said distal end, said at least one port exiting said side wall at a predetermined position relative to said distal end.

2. The apparatus of claim 1 wherein said at least one port includes an entrance orifice at said proximal end of said member and an exit orifice at said distal end of said member, said at least one port extending from said entrance orifice to said exit orifice.

3. The apparatus of claim 1 wherein said member has a central longitudinal axis, said side wall and said inner lumen being centered on said axis, said at least one port extending parallel to said axis.

4. The apparatus of claim 1 wherein said side wall comprises a solid piece of material, said at least one port being bored through said solid piece of material.

5. The apparatus of claim 4 wherein said solid piece of material comprises stainless steel.

6. The apparatus of claim 1 wherein said side wall comprises a tubular inner sleeve positioned coaxially within a tubular outer sleeve, said at least one port being positioned between said inner sleeve and said outer sleeve.

7. The apparatus of claim 6 wherein said at least one port comprises an annular space defined between adjacent portions of said inner sleeve and said outer sleeve.

8. The apparatus of claim 6 wherein said at least one port comprises opposing grooves in adjacent opposing surfaces of said inner and outer sleeves.

9. The apparatus of claim 1 wherein said at least one port is adapted to receive sensor leads for assisting in delivery of the device to the target site.

10. The apparatus of claim 1 wherein said at least one port is adapted to receive a fluid delivery conduit for delivering fluids to the target site.

11. The apparatus of claim 1 wherein said at least one port is adapted to receive a suction conduit for providing suction at the target site.

12. The apparatus of claim 1 further comprising means for assisting delivery of the device to the target site, said assisting means being insertable through said at least one port to position said assisting means at a desired position relative to the target site.

13. The apparatus of claim 1 wherein said member has a curved configuration extending along a curved central axis.

14. An apparatus for facilitating delivery of a device to a target site in a body, said apparatus comprising:
an elongate member having a side wall and at least one inner lumen extending from a proximal end of said member to a distal end of said member, said member being insertable into the body to position said distal end at a predetermined position relative to the target site; and
at least one port comprising a longitudinal bore in said side wall, said at least one port extending from said proximal end toward said distal end, said at least one port exiting said side wall at a predetermined position relative to said distal end.

15. An apparatus for facilitating delivery of a device to a target site in a body, said apparatus comprising:
an elongate tubular member having a longitudinal axis, said member having an axially extending cylindrical side wall with an inner surface defining a cylindrical inner lumen extending from a proximal end of said member to a distal end of said member, said member being insertable into the body to position said distal end at a predetermined position relative to the target site; and
at least one port extending through said side wall in a direction parallel to said axis from said proximal end toward said distal end, said at least one port exiting said side wall at a predetermined position relative to said distal end.

16. An apparatus for facilitating delivery of a device to a target site in a body, said apparatus comprising:
an elongate tubular member having a longitudinal axis, said member including a cylindrical inner sleeve positioned within and extending coaxially with a cylindrical outer sleeve, said inner sleeve having an inner surface defining a cylindrical inner lumen extending from a proximal end of said member to a distal end of said member, said member being insertable into the body to position said distal end at a predetermined position relative to the target site; and
at least one port defined between an inner surface of said outer sleeve and an outer surface of said inner sleeve, said at least one port extending from said proximal end to a predetermined position relative to said distal end.

17. An apparatus for facilitating delivery of a device to a target site in a body, said apparatus comprising:
an elongate cylindrical side wall defining a central lumen extending along an axis from a proximal end of said side wall to a distal end of said side wall; and
at least one port extending within said side wall in a direction parallel to said axis from said proximal end toward said distal end, said at least one port exiting said side wall at a predetermined position relative to said distal end.
